(19) United States
(12) Patent Application Publication
(10) Pub. No.: US 2011/0077535 A1
(43) Pub. Date: Mar. 31, 2011

(54) APPARATUS AND METHOD FOR DIGITAL SPHYGMOMANOMETER

(75) Inventors: Yunquan Chen, Delta (CA); Yun Xu, Beijing (CN)

(73) Assignee: PRECISION MEDICAL TECHNOLOGY LTD., Beijing (CN)

(21) Appl. No.: 12/994,904

(22) PCT Filed: May 27, 2009
(86) PCT No.: PCT/CN2009/072020

§ 371 Date: Nov. 29, 2010

(30) Foreign Application Priority Data
May 27, 2008 (CN) 200810113187.0

(51) Int. Cl. A61B 5/0225

(52) U.S. Cl. 600/493

(57) ABSTRACT

A digital sphygmomanometer comprises a manual pump (22) for inflating an inflatable cuff (24), a pressure sensor (26) for measuring the pressure in said inflatable cuff (24), a digital display (34) for displaying the pressure in said inflatable cuff (24), and a MPU (32) for processing the pressure signal and for controlling the display of the pressure signal. A method for a digital sphygmomanometer comprises procedures embedded in said MPU (32) for detecting the phases of the blood pressure measurement process and for display the pressure in said inflatable cuff (24) in different ways to allow displayed pressure values staying long enough for operator to read while ensuring sufficient display accuracy for the purpose of accurate blood pressure measurement.

### TECHNICAL FIELD

This invention is related to apparatus and method for a digital sphygmomanometer. In particular, the invention is related to the use of a pressure sensor for measuring the pressure and to the display of the pressure in digital forms via a variety of selective methods under the control of a micro-processor or micro-controller unit (MPU).

### BACKGROUND

Existing medical sphygmomanometers include mercury sphygmomanometers, aneroid sphygmomanometers, electronic sphygmomanometers that simulate the mercury pressure display of a mercury sphygmomanometer, and electronic sphygmomanometers that simulate the needle pressure display of an aneroid sphygmomanometer. When using a medical sphygmomanometer to measure the blood pressure of subject, a well trained operator, such as a doctor or nurse, first manually pumps in multiple inflation cycles the pressure in an inflatable cuff to a target pressure, and then, controls a manual valve to deflate the inflatable cuff. Each inflation cycle is a squeeze of the pump to an ending point before releasing the pump. In the deflation phase, the operator reads the pressure value in the inflatable cuff while at the same time uses a stethoscope to listen to the Korotkoff sounds to determine the systolic (high) and diastolic (low) pressure of the subject. The existing medical sphygmomanometers have one or more of the following shortcomings: 1) mercury is one of the three most toxic elements on the earth, which causes serious environmental pollution and has serious impact on people's health; 2) due to the characteristics of metal material used in aneroid sphygmomanometers, they are easy to lose calibration. Therefore, it is necessary to calibrate aneroid sphygmomanometers on a regular basis; 3) simulation of mercury or aneroid sphygmomanometers either has high cost or is of low quality; 4) when using sphygmomanometers discussed above, an operator tends to have a bias toward a number that is a multiple of 5 or 10. For example: when the reading is 147, many operators will read it as 150, resulting in measurement error.

The existing automatic electronic sphygmomanometers generally measure the systolic and diastolic blood pressure by the oscillometric method. Practice shows that the oscillometric method is not always accurate in blood pressure measurement.

### CONTENT OF INVENTION

Recognizing the deficiencies and shortcomings of existing technologies, the inventors analyzed the process of blood pressure measurement by operators and found the following key points:
1) In the inflation phase of blood pressure measurement, the accuracy of pressure measurement is not required to be high. For example, a 5 mmHg reading error in the inflation phase will not have any impact on blood pressure measurement.
2) In the deflation phase of blood pressure measurement, due to a variety of failure causes including inadequate initial inflation and systolic pressure measurement failure, the operator sometimes needs to go back to the inflation phase from the deflation phase.
3) In the deflation phase of blood pressure measurement, operators need to visually determine the deflation rate in order to adjust the rate of deflation to avoid measurement error caused by excessively high deflation rate.
4) In the deflation phase of blood pressure measurement, the time when accurate pressure measurement is required is when the Korotkoff sounds occur. Pressure readings between two Korotkoff sounds are not important.

The inventors did an experiment on healthy subjects in reading speed from constantly updating integers simulating pressure readings on a LCD display. The updating cycle of the displayed integers was under the control of the operator. Through the trial we found that when reading time was limited, if the displayed integers on the LCD display were increased or decreased by the same simulated pressure interval each time, the readings were easier to read because they could be foreseen than if the displayed integers were increased or decreased by a different interval each time. In addition, we also found that readings of a multiple of 5 or 10 were easier to read than other integers. In particular, when the readings were 10 or a multiple of 10, it was the easiest to read. What is more, we found that when the readings on the LCD were increased or decreased by the same simulated pressure interval each time, the rate of increase or decrease could be intuitively determined. Moreover, we also found that when the display update cycle of integers was less than or equal to 0.5 seconds, the integers were difficult to read. Only when the display update cycle was more than 0.6 seconds long, the integers could be read accurately and reliably. In addition, we also found that the most accurate and reliable display update cycle for reading integers was 1 second or longer.

In another experiment, the inventors found that for a typical manual inflation system such as that of a mercury sphygmomanometer, an inflation cycle, which was one squeezing stroke of the manual pump, was greater than 0.5 seconds, usually 1 second or longer. In the experiment the inventors also found that, in the inflation process of a manual inflation system that included a manual pump and a connected inflatable cuff, when the squeeze ending point of the manual pump was reached, the air pressure in the inflatable cuff would have a number of characteristics including the occurrence of a peak pressure and a pressure change rate of zero or negative.

In an experiment related to the squeeze ending point of a manual inflation pump, the inventors found that, utilizing the characteristic that the air pressure in the inflatable cuff would reach a peak pressure when the manual pump was squeezed to an ending point, the ending point of the manual pump could be determined by detecting the peak pressure in the inflatable cuff. Utilizing the characteristic that the air pressure in the inflatable cuff would change from a positive rate of pressure change to a zero or negative rate of pressure change when the manual pump was squeezed to an ending point, the ending point of the manual pump could also be determined by determining whether the rate of pressure change in the inflatable cuff had become zero or negative.

In an experiment on pressure display and Korotkoff sounds, we found that when the pressure in the inflatable cuff was displayed immediately after the detection of the rising slope of a pulse signal from the artery and was maintained until the detection of the next pulse, the display of the pressure value and the occurrence of the Korotkoff sounds appeared in an almost synchronized manner to the ears of the operators, which facilitated the reading of the displayed pressure value at the time when Korotkoff sounds occurred. On the other hand, when a pulse was detected at its peak pressure region or at the descending slope of the pulse, pressure display at the detection of the pulse and the Korotkoff sound became a bit desynchronized, making reading the displayed pressure value at the time of occurrence of Korotkoff sound difficult .

This invention provides a digital sphygmomanometer that uses a digital display to display pressure. Programs installed in a micro-processor or micro-controller unit (MPU) in said sphygmomanometer continuously detect certain pressure events occurring in the inflatable cuff during blood pressure measurement and determine a number of phases of blood pressure measurement according to those pressure events. The various phases of blood pressure measurement include inflation and deflation phases. The deflation phase is further divided into a non-pulsed deflation phase and a pulsed deflation phase. The programs use different pressure display methods to display pressure readings in different phases of blood pressure measurement.

When it is detected that a pressure increase is more than a certain amount of value within a certain period of time, the programs determine that the operator is operating the manual pump to inflate the inflatable cuff, and therefore the blood pressure measurement is in the inflation phase. Under the control of the MPU, the pressure values or their approximate values are displayed only when certain pressure events happen such as when the squeeze ending point of the manual pump or a pressure that is a multiple of 10 mmHg is reached. When neither a pressure increase of more than a certain amount of value within a certain period of time nor a pulse signal is detected, the programs determine that the blood pressure measurement is in the non-pulsed deflation phase. Under the control of the MPU, the pressure values are displayed with a constant interval of pressure change between displayed values or with other similar method. When a pressure increase of more than a certain amount of value within a certain period of time is not detected, but a pulse signal is detected, the programs determine that the blood pressure measurement is in the pulsed deflation phase. Under the control of the MPU, only pressure values at the times of pulse detection are displayed.

There are an inflation detection program, an inflation pressure display program, a pulse signal detection program, a pulsed deflation pressure display program and a non-pulsed deflation pressure display program embedded in said MPU.

Said inflation detection program detects any possible inflation to said inflatable cuff by an operator at anytime of blood pressure measurement, and starts or maintains the inflation pressure display program when it is determined that said inflatable cuff is in the inflation phase. Said pulse signal detection program is used in the deflation phase of the inflatable cuff. The pulse signal detection program detects pulse signals in the pressure in said inflatable cuff, and starts or maintains the pulsed deflation pressure display program when pulse signals are detected. When the pressure in the inflatable cuff is in the default non-pulsed deflation phase, the non-pulsed deflation pressure display program is started or maintained.

Said inflation detection program continuously detects the pressure in the inflatable cuff throughout the entire blood pressure measurement process, and determines whether the pressure in the inflatable cuff is in the inflation phase. The criteria for determining the inflation phase are as follows: In a given period of time, the pressure inside the inflatable cuff increases a given amount of value. For example, the given period of time may be one second and the given amount of value of the pressure increase may be 5 mmHg.

The inflation pressure display program may use any one of the following methods to display the pressure in the inflatable cuff during the inflation phase: 1) to display the pressure value or its approximate value in the inflatable cuff when the squeeze ending point of the manual pump is detected, which may be determined by detecting the pressure peak or the rate of pressure change in the inflatable cuff during the inflation phase; 2) to display the pressure value or its approximate value in the inflatable cuff when a pressure increase of 10 mmHg or a multiple of other easy-to-read numbers is detected; 3) to display the pressure value in the inflatable cuff when the pressure in the inflatable cuff is a multiple of 10 mmHg or a multiple of other easy-to-read numbers. Practical examples of other easy-to-read pressure values are 5 mmHg and 1 kPa.

The non-pulsed deflation pressure display program displays pressure values or their approximate values with a constant interval of pressure decrease of at least 0.2 kPa or 2 mmHg, or a maximum of 0.5 kPa or 4 mmHg. Practical constant intervals of pressure are 0.2 kPa, 2 mmHg, 3 mmHg, 0.5 kPa, or 4 mmHg Said approximate values are the closest values to the real pressure values in the inflatable cuff that meet said constant interval requirements.

The pulse signal detection program detects the pulse signal in the pressure in the inflatable cuff during the deflation phase. The detection may be local peak pressure detection. When a local peak pressure reaches a given value, it is determined that a pulse signal is detected. Preferably, the detection of a pulse signal is carried out as a detection of the rising slope of a pulse signal. When a pulse signal is detected, the pulsed deflation pressure display program is started or maintained. At the same time, the inflation detection program and the pulse signal detection program continue to be carried out. If in a given period of time, for example, in 1.5-2.0 seconds, there is no pulse signal detected, the program automatically transfers to the default non-pulsed deflation phase, and starts up non-pulsed deflation display program. At the same time, the pulse signal detection program and inflation detection program continue to be carried out.

The pulsed deflation pressure display program may, upon detection of a pulse signal, display the pressure in the inflatable cuff and maintains the displayed value without updating the displayed value with the pressure value of the inflatable cuff until the next pulse is detected or until after a certain period of time has elapsed without detection of the next pulse signal. Said certain period of time may be chosen between 1.5-2.0 seconds.

This invention also provides a pressure display method for a digital sphygmomanometer. Said sphygmomanometer includes a manual pump, a deflation valve, an inflatable cuff connected with the manual pump and deflation valve, a pressure sensor connected with the inflatable cuff, electronic circuits connected with the pressure sensor and a MPU as well as a digital display connected with the MPU. There are an inflation detection program, inflation pressure display program, pulsed signal detection program, pulsed deflation pressure display program and a non-pulsed deflation pressure display programs embedded in said MPU. The pressure values in the inflatable cuff or their approximate values are displayed on the digital display.

One aspect of the pressure display method is to display the pressure value in the inflatable cuff on the display immediately when a pulse signal in the pressure in the inflatable cuff is detected, preferably by the detection of the rising slope of a pulse signal, and to maintain the displayed value and not to update the displayed pressure value until the next pulse is detected or after a certain period of time has elapsed without detecting the next pulse signal. Said certain period of time may be chosen between 1.5-2.0 seconds.

Another aspect of the pressure display method is to display pressure values or their approximate values in a way in which each displayed pressure value is decreased from the previously displayed value by an constant interval of at least 0.2 kPa or 2 mmHg, or a maximum of 0.5 kPa or 4 mmHg when the blood pressure measurement is in the non-pulsed deflation phase.

Another aspect of the pressure display method is to display the pressure value in the inflatable cuff or its approximate value when the manual pump is squeezed to a squeeze ending point or when the pressure in the inflatable cuff is inflated over a value that is a multiple of 10 mmHg or 5 mmHg or 1.0 kPa.

Further aspects of the invention and features of specific embodiments of the invention are described below.

### BRIEF DESCRIPTION OF THE DRAWINGS

In drawings which illustrate non-limiting embodiments of the invention,

FIG. 1 is a hardware block diagram of the digital sphygmomanometer;

FIG. 2 is a software flowchart of the digital sphygmomanometer;

FIG. 3 illustrates the division of phases and some important pressure events during a cycle of blood pressure measurement;

FIG. 4 illustrates the use of a digital sphygmomanometer;

FIG. 5 illustrates the rising slope, peak region and descending slope of a pulse signal.

The marks used in the drawings are as followed:

20—stethoscope, 22—manual pump, 23—valve, 24—inflatable cuff, 26—pressure sensor, 28—differential amplifier, 30—A/D converter, 32—MPU, 34—display, 42—inflation phase, 44—deflation phase, 46—non-pulsed deflation phase, 48—pulsed deflation phase, 52—initialization, 54—data acquisition, 56—inflation phase determination, 58—inflation peak pressure measurement, 60—inflation peak pressure determination, 62—calculation of the approximate value of the inflation pressure display, 64—pressure display update, 70—pulse signal detection, 72—pulse signal determination, 74—acquisition of pressure value for display in pulsed deflation phase, 76—calculation of the approximate value of pressure for display in the non-pulsed deflation phase.

### DETAILED DESCRIPTION OF EMBODIMENT

Throughout the following description, specific details are set forth in order to provide a more thorough understanding of the invention. However, the invention may be practiced without these particulars. In some cases, well known elements have not been shown or described in detail to avoid unnecessarily obscuring the invention. Accordingly, the specification and drawings are to be regarded in an illustrative, rather than a restrictive, sense.

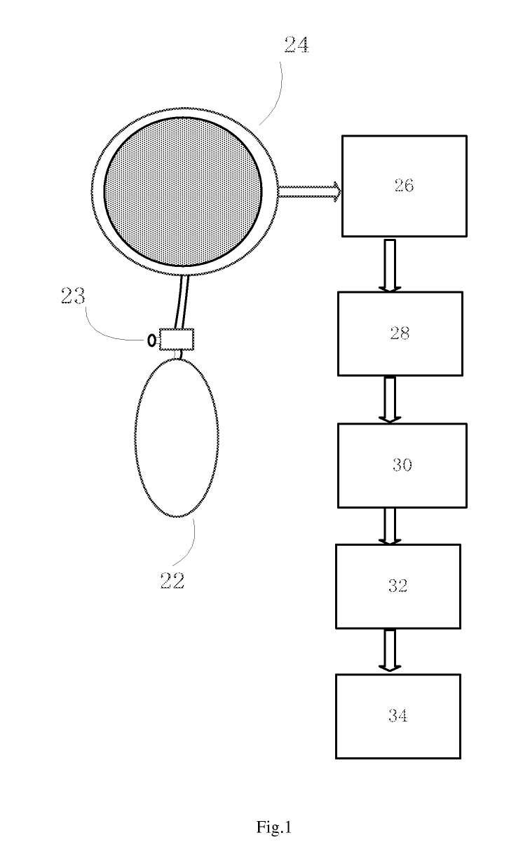

With the attached drawings (FIG. 1-FIG. 5), detailed description about the invention is as follows.

As shown in FIG. 1, a digital sphygmomanometer includes a manual pump 22, an inflatable cuff 24, a display 34 which is represented by an LCD, a measurement system, and a control system. The measurement includes pressure sensor 26, which usually is an impedance bridge, a differential amplifier 28, an A/D converter 30, and a micro-processor or micro-controller unit (MPU) 32. The measurement system may also include MPU 32 and an integrated sensor that has a pressure sensor, a differential amplifier and A/D convert all on the same chip. Similarly, the A/D converter 30 can also be integrated in MPU 32. The control system includes MPU 32 and the display under its control. Both the measurement and control systems include MPU 32. The programs in MPU 32 consists of detection and display programs including inflation detection, inflation pressure display, pulse signal detection, pulsed deflation pressure display, and non-pulsed deflation pressure display. The non-pulsed deflation pressure display program is set as the default pressure display program.

Manual pump 22 is a hand-operated pump, but it may also be a foot-operated pump or some other types of pumps. Inflatable cuff 24 is usually applied onto the arm, but it may also be applied onto the thigh, leg, wrist etc. Similarly, an inflatable air cushion may also be applied to the skin close to an artery for measurement of blood pressure.

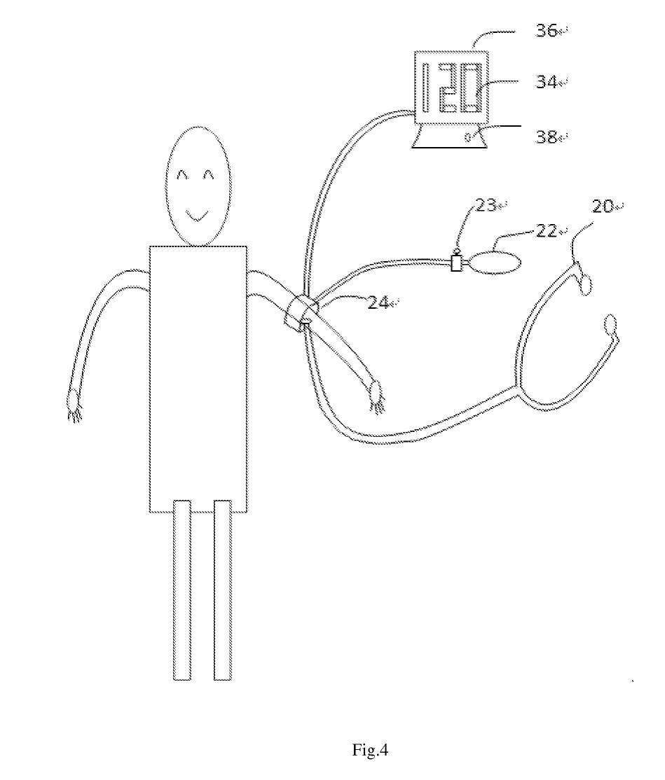

As shown in FIG. 4, when measuring the blood pressure, the operator starts the electronic part of the digital sphygmomanometer through the power switch 38, then, inflates the inflatable cuff 24 by squeezing the manual pump 22. The air pressure in the inflatable cuff 24 is transformed into a pressure signal through the pressure sensor 26 shown in FIG. 1. After amplification through differential amplifier 28 shown in FIG. 1, the pressure signal shall be converted to a digital signal by the A/D converter 30 and acquired by the MPU 32. The digital pressure is calculated and processed by the programs in the MPU 32, and then the pressure may be displayed on the display 34 in numerical values in a variety of ways. When pressure in the inflatable cuff 24 reaches a target pressure (usually 20-40 mmHg higher than the systolic pressure or high pressure of the subject whose blood pressure is being measured), the operator releases valve 23 and deflates the inflatable cuff 24 slowly. While watching the display 34 displaying the pressure values in a variety ways, the operator measures the blood pressure by listening to the Korotkoff sounds with a stethoscope. Said digital sphygmomanometer may further include an automatic deflation valve. Slow deflation of the inflatable cuff 24 may be achieved by the automatic deflation valve.

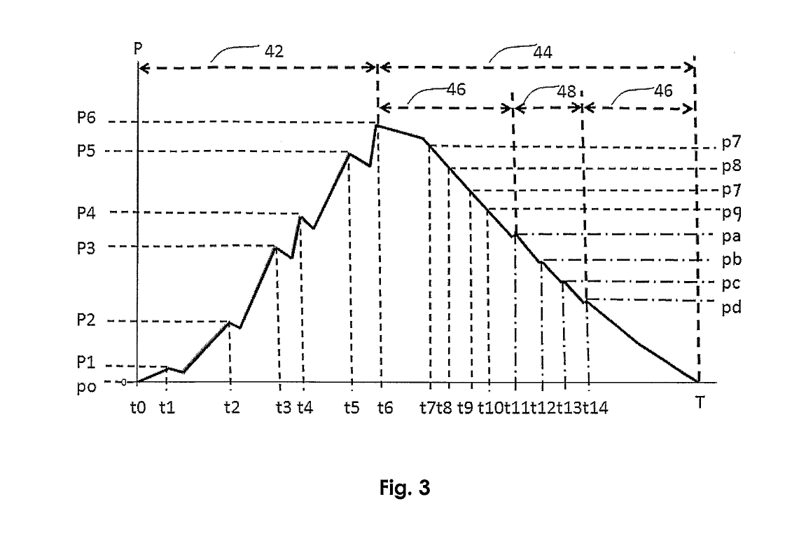

As shown in FIG. 3, a blood pressure measurement cycle may be divided into two main phases: inflation phase 42 and deflation phase 44. Deflation phase 44 may further be divided into non-pulsed deflation phase 46 and pulsed deflation phase 48. Pulsed deflation phase 48 is a period of time when pulse signals are detected by MPU 32 from the pressure signal during deflation phase 44. The ways in which the pressure is displayed on display 34 is decided based on the determination of inflation/deflation phases during the cycle of blood pressure measurement, which is mainly done by the inflation detection program and the pulse signal detection program. Different ways of pressure display on display 34 is done primarily by the inflation pressure display program, pulsed deflation pressure display program and non-pulsed deflation pressure display program.

During inflation phase 42, the manual pump 22 shown in FIG. 1 is squeezed, so that air is pushed into the inflatable cuff 24. After air in pump 22 has been squeezed into the inflatable cuff 24, the manual pump 22 is released, it restores its original state and air outside pump 22 goes into the pump 22 at the same time filling the entire space inside of it. Each squeezing movement is named as pump squeezing. At each squeeze ending point of the pump (As the examples of t1-t6 shown in FIG. 3), the pressure in the inflatable cuff 24 reaches the peak pressure (As the examples of p1-p6 shown in FIG. 3).After several times of repeated pump squeezing (six times in the example of FIG. 3), the pressure in the inflatable cuff 24 reaches the target pressure.

When the pressure in the inflatable cuff 24 reaches the target pressure that the operator needs, the operator shall stop squeezing the manual pump 22 and deflates the inflatable cuff 24 through the valve 23. The blood pressure measurement enters the deflation phase. When in deflation phase, the display program in MPU 32 changes to the default pressure display of non-pulsed deflation pressure display immediately, and starts the pulse signal detection program at the same time.

During deflation phase 44, the valve 23 is opened partially, so that air in the inflatable cuff 24 is released out of it slowly, and the pressure in the inflatable cuff 24 is lowered. When the pressure in the inflatable cuff 24 is lowered to a certain value, the pulse signal detection program in the MPU 32 shall begin to detect the pulse signals in the pressure signal of the inflatable cuff 24. When the pressure in the inflatable cuff 24 is lowered to another certain value, the pulse signal said above disappears. In deflation phase 44, the period of time before the first time a pulse is detected (as the example pa shown in FIG. 3) and the period of time after the last time a pulse is detected (as the example pd shown in FIG. 3) is defined as the non-pulsed deflation phase 46. The period of time during which pulse signals are detected (as the example t11-t14 shown in FIG. 3) is defined as the pulsed deflation phase 48. In deflation phase 44, the measurement of blood pressure is realized by using the commonly used Korotkoff sound method or other methods.

During the deflation phase of the blood pressure measurement, the operator sometimes needs to go back to inflation before deflation is completed because of a number of reasons including that the initial inflation may not be enough and that detection of the systolic pressure fails.

The inflation detection program in MPU 32 may detect possible inflation to inflatable cuff 24 by an operator at any time during blood pressure measurement, and start or maintain the inflation pressure display program in MPU 32 when the pressure in inflatable cuff 24 is in inflation phase. When pressure in the inflatable cuff 24 belongs to deflation phase 44, the pulsed signal detection program in MPU 32 detects pulse signals in inflatable cuff 24 and starts or maintains the pulsed deflation pressure display program in MPU 32. When pressure in the inflatable cuff 24 belongs to non-pulsed deflation phase 46, the deflation pressure display program in MPU 32 starts or maintains the non-pulsed deflation pressure display program.

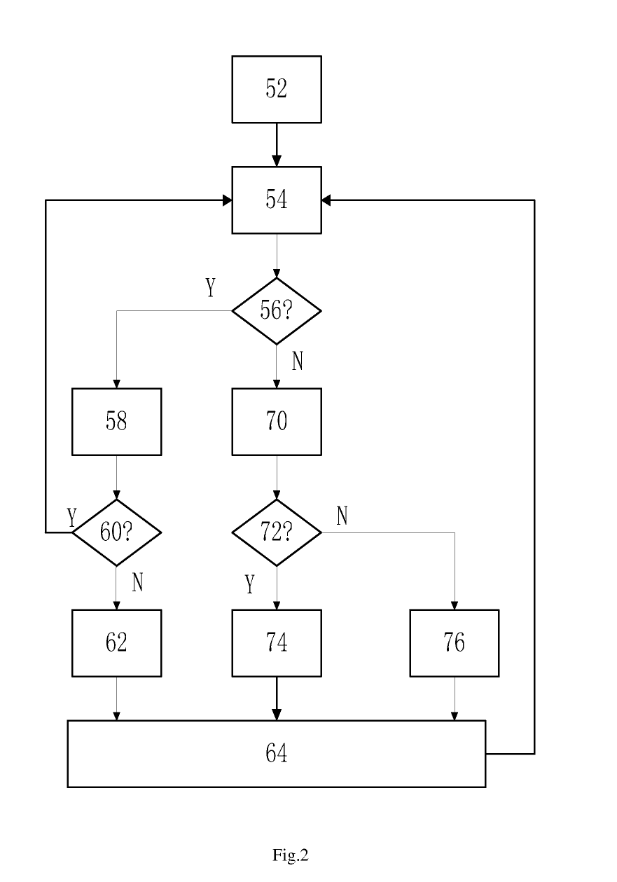

The inflation detection program, inflation pressure display program, pulse detection program and the pulsed deflation pressure display program embedded in the MPU 32 are overlapping and indivisible parts of a whole unit. FIG. 2 illustrates an example of the programs in MPU 32, which include the following major steps:
a) Initialization 52 includes updating the display on display 34 shown in FIG. 1 and recording the updated time of the display 34. The initial values are usually zero. After initialization, the inflation detection program and inflation pressure display program in b) to g) start to run.
b) Data acquisition 54 includes acquiring the pressure value P(t) in inflatable cuff 24 at present time t
c) Inflation phase determination 56 compares the present pressure P(t) with the previous pressure P (t−ΔT) , where ΔT is between 0.5 and 1.5 second, preferably 1 second. If P(t) is not larger than P (t−ΔT) plus a given pressure increase value, then the program in the MPU 32 judges that the inflation phase 42 has finished and that the pressure in inflatable cuff 24 is in deflation phase 44 as shown in FIG. 3. In this case, the program shall go to step i), entering the pulse signal detection program; If P (t) is larger than P (t−ΔT) plus a given pressure increase value, the program judges that the pressure in inflatable cuff 24 is in inflation phase, and continues to run the inflation pressure display program from d) to g). The given pressure increase value may be between 5 mmHg and 10 mmHg.
d) Inflation peak pressure measurement 58 calculates dP1=P (t−Δt)−P (t−2 Δt) and dP2=P (t−Δt)−P (t) where Δt is between 0.05 and 0.2 seconds and preferably 0.1 seconds.
e) Inflation peak pressure determination 60 compares dP1 and dP2 with zero. If dP1 is large than zero and dP2 is large than or equal to zero, a peak pressure is detected and the program shall go on to step f) to update the pressure displayed on display 34 shown in FIG. 2; otherwise, a peak pressure is not detected and the program shall go back to step b), skipping updating the pressure displayed on display 34.
f) Inflation display pressure approximate value calculation 62 calculates an approximate value Pd that is a multiple of 10 and the closest value to the real value P (t−Δt).
g) Pressure display update 64 updates the displayed value on display 34 shown in FIG. 1 with the calculated approximate value Pd.
h) Keep repeating steps b) to g) until step c) judges that the inflation 42 is finished.
i) Pulse signal detection 70 uses dP=P (t−Δt)−P (t−2 Δt)+P(t))/2 to calculate local peak pressure dP to search for pulse signals where Δt is between 0.05 and 0.2 seconds and preferably 0.1 seconds.
j) Pulse deflation phase determination 72 compares dP with dp, where dp is between 0.2-1.0 mmHg, preferably 0.6 mmHg. If dP is large than dp, a pulse signal is detected; otherwise, a pulse signal is not detected. If at least one pulse signal is detected within a given period of time, it is determined that the pressure in inflatable cuff 24 is in the pulsed deflation phase 48 shown in FIG. 3. Otherwise, it is in the non-pulsed deflation phase 46. Said given time is between 1.5 and 2 seconds. If it is in pulsed deflation phase 48, the program goes to pulsed deflation pressure display program l) and m). Otherwise the program goes to non-pulsed deflation pressure display program k) and m).
k) Non-pulsed deflation pressure display approximate value calculation 76 calculates the value Pd that is a multiple of 2 and the closest value to the real value P(t). Then, the program goes to step m).
l) Pulsed deflation pressure display value acquisition 74 acquires pressure P(tp) which is the pressure in inflatable cuff 24 at the time tp when the latest pulse signal is detected.
m) Pressure display update 64 updates the displayed value on display 34 shown in FIG. 1 with Pd or P(tp), and then the program goes back to step b), starting a new cycle of inflation detection, pulse signal detection and related pressure display programs.

The goal of the inflatable cuff 24's pressure display value acquisition 74, display pressure approximate value calculation 62, 76 and pressure display update 64 is to display a value in digital form on display 34 so that the digital value and the way in which the digital value is displayed is easy for the operator to read. In addition, the digital value must be accurate enough to represent the real pressure in the inflatable cuff 24 according to the pressure display requirements in different phase of blood pressure measurement. There are other methods that are easy for operators to read and that are accurate enough to display pressure values. For example, when the unit of pressure value is mmHg, in approximate calculation 62, the approximate value may be a multiple of 5; when the unit of pressure value is kPa, the approximate value may be a multiple 1 or 2. In approximation calculation 76, when the unit of pressure value is mmHg, the approximate value may be a multiple of 3 or 4; when the unit of pressure value is kPa, the approximation value may be a multiple of 0.2 or 0.5.

We may also use other methods for inflation peak pressure measurement 58 and inflation peak pressure determination 60. For example, we may calculate dP=P (t−Δt)−(P(t−2 Δt)+P(t))/2, where is between 0.05 to 0.2 seconds. If dP is larger than 0, then it is indicated that a pressure peak is detected.

Inflation peak pressure measurement 58 is a method of detecting the squeeze ending point (t1, t2, t3, t4, t5, t6) shown in FIG. 3 of manual pump 22 shown in FIG. 1. There are other methods for detecting the squeeze ending point of the inflatable cuff. For example, in inflation phase 42 shown in FIG. 3, after going through a certain point of pump squeeze, there is a pressure drop or lack of pressure increase in the inflatable cuff. This point may be regarded as the pump squeeze ending point. The rate of pressure change may be determined as follows: dP=P(t)−P(t-−Δt), where P is pressure value, t is time, Δt is between 0.05 to 0.2 seconds, preferably 0.1 seconds. If dP is smaller or equal to 0, then, it is indicated that a pump squeeze ending point is detected. If dP is larger than 0, then a pump squeeze ending point is detected.

The detection of the squeeze ending point of the pump in inflation phase 42 is a method for determining relevant pressure events that are suitable for updating the displayed pressure value on display 34. There are other methods for doing the same thing. For example, a relevant pressure event may also be a pressure increase of 5 mmHg or more of the current pressure in inflatable cuff 24 compared to the pressure at the time when the displayed value on display 34 was last updated. It is preferable that the pressure increase is 10 mmHg or 1 kPa. It is the most preferable that the displayed pressure values are multiples of 10 mmHg or 1 kPa. These displayed values are the easiest for operators to read. The relevant pressure events may also further include relevant time events. For example, a relevant pressure event may be the combination of a pressure increase of 5 mmHg or more and elapsed time of 0.5 seconds or more since the last update of display 34. We may, of course, use time events alone for updating displayed values on display 34. For example, we may update the displayed values on display 34 when 0.5 seconds or more has elapsed since the last update of the displayed value on display 34.

In inflation phase 42, the calculation of the pressure values or their approximate values displayed on display 34 is achieved by the inflation display program in MPU 32. A good example is that the pressure display starting point is 0 mmHg, and the approximate value is a multiple of 10 mmHg. As shown in FIG. 1, the manual pump is squeezed by an operator so that air is pushed into inflatable cuff 24. In the process of the air inside the manual pump 22 being squeezed into inflatable cuff 24, the pressure in inflatable cuff 24 is acquired into MPU 32, which detects the rising of pressure in inflatable cuff 24. Therefore, the pressure in inflatable cuff 24 is determined to be in inflation phase 42. MPU 32 calculates the approximate value of the pressure obtained in inflatable cuff 24 as a multiple of 10 mmHg using the rounding principle of discarding values of less than 5 and rounding up values of 5 or larger to 10 for the last digit of an integer. Then, the calculated approximate value is compared with the displayed value on display 34. If the approximate value is increased by 10 mmHg, then, the MPU 32 shall update the displayed value on display 34 with the current approximate value. Otherwise, the displayed value on display 34 shall not be changed. Using this method, the displayed value in display 34 will be updated once for each 10 mmHg increase in pressure, and the maximum error of the displayed pressure is 5 mmHg when compared with the pressure in inflatable cuff 24. This is accurate enough for pressure monitoring in inflation phase 42.

In non-pulsed deflation phase 46, MPU 32 carries out the non-pulsed deflation pressure display program. For example, it displays on display 34 pressure values or their approximate values in inflatable cuff 24 in a way in which the displayed values have a constant interval of pressure decrease, such as examples p5, p6, p7, p8 shown in FIG. 3. The constant interval of pressure may be 2 mmHg, 0.2 kPa, 3 mmHg, 0.5 kPa, or 4 mmHg. The end point of pressure display is preferably 0 mmHg, thus, the displayed pressure values or their approximate values are a multiple of the constant interval of pressure. For example, the pressure values in inflatable cuff displayed on display 34 are 180 mmHg, 178 mmHg, 176 mmHg, 174 mmHg and so on. These pressure display values are multiples of the constant pressure interval 2 mmHg. They are easy for an operator to read. At the same time, since the decreases have a constant pressure interval, the update frequency of the displayed values is proportional to the rate of pressure change in inflatable cuff 24, so that the operator may intuitively judge the rate of pressure change in inflatable cuff 24 according to the update frequency of the display values. This is similar to the situation where the operator intuitively judges the rate of pressure change in inflatable cuff 24 according to the rate of drop of a mercury column of a mercury sphygmomanometer.

In non-pulsed deflation phase 46, the calculation of the pressure values or their approximate values displayed on display 34 is achieved by the non-pulsed deflation pressure display program in MPU 32. A good example is that the pressure display starting point is 0 mmHg, and the approximate value is a multiple of 2 mmHg. As shown in FIG. 1, when the manual pump 22 is stopped by the operator, the operator will gradually turn on valve 23, so that the air in inflatable cuff 24 will be released and the pressure in inflatable cuff 24 will decrease. In the process of deflation, the pressure in inflatable cuff 24 is acquired into MPU 32, which detects the decreasing of pressure in inflatable cuff 24, but does not detect pulse signals. Therefore, the pressure in inflatable cuff 24 is determined to be in non-pulsed deflation phase 46. MPU 32 calculates the approximate value of the pressure obtained in inflatable cuff 24 as a multiple of 2 mmHg using the rounding principle (that is to discard pressure decrease values of less than half of the constant pressure interval and round up pressure decrease values of a half or more of the constant pressure interval to the full constant pressure interval). Then, the calculated approximate value is compared with the displayed value on display 34. If the approximate value is decreased by 2 mmHg, then, the MPU 32 shall update the displayed value on display 34 with the current approximate value. Otherwise, the displayed value on display 34 shall not be changed. Using this method, the maximum error of the displayed pressure is 1 mmHg when compared with the pressure in inflatable cuff 24. This is accurate enough for pressure monitoring in non-pulsed deflation phase 46.

Since updating display 34 with the same value as displayed on display 34 is equivalent to not updating the displayed value on display 34 at all, the method of updating the displayed value on display 34 after comparison may be simplified to updating the displayed value on display 34 with the approximate value every time.

In pulsed deflation phase 48, when the pulse detection program in MPU 32 detects a pulse signal in the pressure signal in inflatable cuff 24, the pulsed deflation pressure display program in MPU 32 immediately displays the pressure value in inflatable cuff 24 at the time in which the pulse is detected, and maintains the displayed value until the next pulse signal comes or the pulsed deflation phase comes to an end before updating the display again. Since a person's pulse rate is typically between 40 and 120 beats per minute, this method of updating displayed pressure for each pulse signal allows the operator to have from 0.5 to 1.5 seconds to read the displayed pressure, and the displayed pressure value is exactly the one that needs to be read precisely.

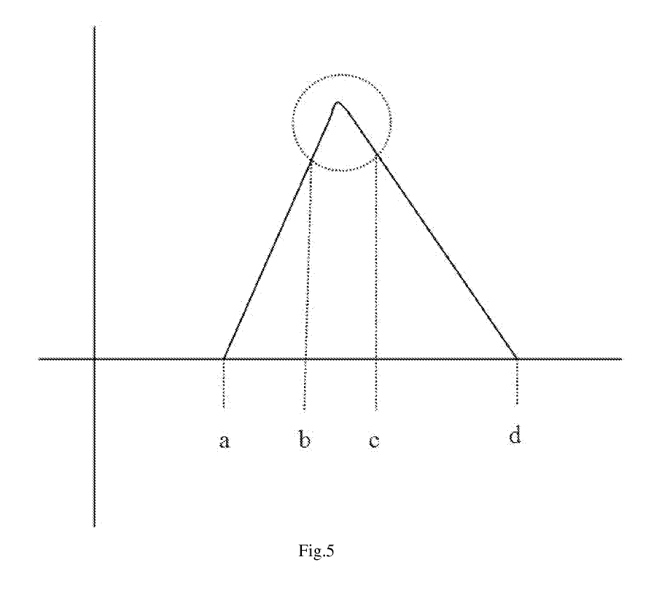

As shown in FIG. 5 a pulse signal may include a rising slope, a peak region and a descending slope. Pulse detection may be achieved by searching a local peak pressure as described in pulse signal detection 70 discussed above. More preferably, pulse detection is achieved by the detection of the rising slope of the pulse signal. This may be done by calculating the first derivative of the pressure signal and deducting the general slope of the pressure signal to obtain a pure first derivative of the pulse signal. On the rising slope of the pulse signal, the first derivative is a positive number. By comparing it with a threshold value, preferably between 3-8 mmHg/s, we can detect the rising slope of a pulse.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

1. A digital sphygmomanometer for measurement of blood pressure of a subject, said sphygmomanometer comprising:
A) An inflatable cuff for occluding an artery of a subject;
B) A manual pump for inflating said inflatable cuff;
C) A pressure sensor for sensing a pressure in said inflatable cuff;
D) A digital display for displaying a pressure in said inflatable cuff; and
E) A MPU for processing pressure signals from said inflatable cuff and for controlling said digital display, wherein said MPU automatically detects one of an inflation phase, a non-pulsed deflation phase and a pulsed deflation phase of blood pressure measurement with said digital sphygmomanometer, and controls said digital display to display said pressure in said inflatable cuff with a corresponding one of inflation, non-pulsed deflation and pulsed deflation display procedures.

2. A digital sphygmomanometer as in claim 1, wherein said inflation display procedure comprises displaying said pressure in said inflatable cuff in an approximate value of said pressure, said approximated value being a multiple of a given pressure value, said given pressure value being one of 5 mmHg, 10 mmHg, 1.0 kPa and 2.0 kPa.

3. A digital sphygmomanometer as in claim 1, wherein said inflation display procedure comprises the following steps:
A) Detecting an ending point of an inflation squeeze of said manual pump; and
B) Displaying the pressure in said inflatable cuff at the time of detection of said ending point.

4. A digital sphygmomanometer as in claim 3, wherein said pressure in said inflatable cuff at the time of detection of said ending point is displayed in an approximate value of said pressure, said approximate value being a multiple of a given pressure value, said given pressure value being one of 5 mmHg, 10 mmHg, 1.0 kPa and 2.0 kPa.

5. A digital sphygmomanometer as in claim 1, wherein said non-pulsed deflation display procedure comprises displaying said pressure in said inflatable cuff in an approximate value of said pressure, said approximate value being the last displayed value minus a constant pressure interval, said constant pressure interval being one of 2 mmHg, 3 mmHg, 4 mmHg, 0.2 kPa and 0.5 kPa.

6. A digital sphygmomanometer as in claim 1, wherein said pulsed deflation display procedure comprises displaying a pressure obtained in said inflatable cuff at the time when a pulse is detected and keeping the displayed pressure value on said digital display until the next pulse is detected or until a given period of time has elapsed without detecting a pulse.

7. A method for displaying a pressure in an inflatable cuff of a digital sphygmomanometer on a digital display of said digital sphygmomanometer for blood pressure measurement, said method comprising the steps of:
A) Under the control of a MPU in said digital sphygmomanometer, automatically detecting one of an inflation phase, a non-pulsed deflation phase and a pulsed deflation phase of said blood pressure measurement with said digital sphygmomanometer, and
B) Displaying said pressure in said inflatable cuff with a corresponding one of inflation, non-pulsed deflation and pulsed deflation display procedures.

8. A method as in claim 7, wherein said inflation display procedure comprises displaying said pressure in said inflatable cuff in an approximate value of said pressure, said approximate value being a multiple of a given pressure value, said given pressure value being one of 5 mmHg, 10 mmHg, 1.0 kPa and 2.0 kPa.

9. A method as in claim 7, wherein said inflation display procedure comprises the steps of:
A) Detecting an ending point of an inflation squeeze of a manual pump of said digital sphygmomanometer; and
B) Displaying said pressure in said inflatable cuff at the time of detection of said ending point.

10. A method as in claim 9, wherein said pressure in said inflatable cuff at said time of detection of said ending point is displayed in an approximate value of said pressure, said approximate value being a multiple of a given pressure value, said given pressure value being one of 5 mmHg, 10 mmHg, 1.0 kPa and 2.0 kPa.

11. A method as in claim 7, wherein said non-pulsed deflation display procedure comprises displaying said pressure in said inflatable cuff in an approximate value of said pressure, said approximate value being the last displayed value minus a constant pressure interval, said constant pressure interval being one of 2 mmHg, 3 mmHg, 4 mmHg, 0.2 kPa and 0.5 kPa.

12. A method as in claim 7, wherein said pulsed deflation display procedure comprises displaying a pressure obtained in said inflatable cuff at the time when a pulse is detected and keeping the displayed pressure value on said digital display until the next pulse is detected or until a given period of time has elapsed without detecting a pulse.

13. A digital sphygmomanometer as in claim 1, wherein said MPU is programmed to detect said inflation phase at any time during said blood pressure measurement when said pressure in said inflatable cuff goes up by a given amount in a given period of time.

14. A digital sphygmomanometer as in claim 1, wherein said pulsed deflation display procedure comprises the steps of
A) Detecting the rising slope of a pulse signal in said pressure in said inflatable cuff of said digital sphygmomanometer during a blood pressure measurement,
B) Upon detection of said rising slope of a pulse signal, obtaining the pressure in said inflatable cuff;
C) Displaying on said digital display said pressure obtained at the time when said rising slope of said pulse is detected; and
D) Keeping the displayed pressure value on said digital display until the rising slope of the next pulse is detected or until a given period of time has elapsed without detecting a pulse.

15. A method as in claim 7, wherein said pulsed deflation procedure comprises the steps of
A) Detecting the rising slope of a pulse signal in said pressure in said inflatable cuff of said digital sphygmomanometer during a blood pressure measurement,
B) Upon detection of said rising slope of a pulse signal, displaying on said digital display the pressure in said inflatable cuff obtained at the time when said rising slope of said pulse is detected; and
C) Keeping the displayed pressure value on said digital display until the rising slope of the next pulse is detected or until a given period of time has elapsed without detecting a pulse.
